(19) United States
(12) Patent Application Publication
(10) Pub. No.: US 2012/0238805 A1
(43) Pub. Date: Sep. 20, 2012

(54) ENDOSCOPIC DEVICE

(75) Inventors: Masayuki IWASAKA, Ashigarakami-gun (JP); Tomohiro Ohki, Ashigarakami-gun (JP)

(73) Assignee: Fujifilm Corporation, Tokyo (JP)

(21) Appl. No.: 13/423,007

(22) Filed: Mar. 16, 2012

(30) Foreign Application Priority Data
Mar. 17, 2011 (JP) JP2011-059201

(51) Int. Cl. A61B 1/018

(52) U.S. Cl. 600/104

(57) ABSTRACT

An endoscopic device provided with an observation window in a distal end face of an insertion part to be inserted into a subject, an observational direction from the observation window being substantially identical with a longitudinal axis of the insertion part, the endoscope comprising:
a concave guide surface disposed in a distal lateral portion of the insertion part so as to extend along a longitudinal direction of the insertion part; and
a bending part configured to be bendable with the guide surface inside, wherein
a treatment instrument fed from a distal end exit of a treatment instrument passage disposed closer to a proximal end of the insertion part than the guide surface is guided along the guide surface when the bending part is bent.

### BACKGROUND OF THE INVENTION

1. Field of the Invention

The present invention relates to an endoscopic device, and specifically to an endoscopic device suitable for an operation on the pancreaticobiliary ductal system.

2. Description of the Related Art

Conventionally, in an operation using an endoscope on the biliary tract or the pancreatic duct (pancreaticobiliary ductal system), when a treatment, such as interior observation or stent placement using a stent delivery system, is performed, a side-viewing endoscope provided with a treatment-instrument passage channel is used.

The side-viewing endoscope has an observational field of view for an observation device at a position lateral to a distal end of an insertion part, and is also laterally provided with a treatment-instrument exit of the treatment-instrument passage channel from which a treatment instrument is fed, so that the treatment instrument is fed in the same direction as the observational field of view (see Japanese Patent Application Laid-Open No. 2007-136044, etc.).

When such a side-viewing endoscope is used to perform an operation on the pancreaticobiliary ductal system, the side-viewing endoscope is inserted to the vicinity of the papilla of Vater, a treatment instrument appropriate for a treatment content is inserted into the treatment instrument passage channel after the papilla of Vater is confirmed with the observation device, and the treatment instrument fed from the treatment instrument exit is inserted into the lumen of the pancreaticobiliary ductal system.

When the inside of the pancreaticobiliary ductal system is observed, a endoscope with a small diameter (child scope) capable of being inserted into the treatment instrument passage (insertion) channel of the side-viewing endoscope is used like Japanese Patent Application Laid-Open No. H11-42207, and the child scope is inserted into the lumen of the pancreaticobiliary ductal system through the treatment instrument passage (insertion) channel of the side-viewing endoscope in the same manner as the treatment instrument.

### SUMMARY OF THE INVENTION

However, in the method of performing an operation on the pancreaticobiliary ductal system using the side-viewing endoscope, as described above, observation and treatment of the inside of the lumen of the pancreaticobiliary ductal system cannot simultaneously be performed, so that the child scope needs to be replaced with the treatment instrument. Sometimes the treatment instrument is also replaced with a different type of treatment instrument. In such a case, repeating insertion and removal of the treatment instruments or the like into and from the lumen of the pancreaticobiliary ductal system in the above method is time-consuming and puts a heavy strain on the operator and the patient. Further, when the treatment instruments are replaced, a method of interposing a guide wire in order to secure an insertion route to the lumen of the pancreaticobiliary ductal system is generally used, but it is also troublesome and time-consuming to insert and put the guide wire, and, providing the guide wire is used, there is the disadvantage that the treatment instrument needs to have a lumen through which the guide wire is passed, which interferes with diameter reduction of the treatment instrument.

On the other hand, if a treatment instrument passage channel is disposed inside the insertion part having a small diameter so that the treatment instrument passage channel can directly be inserted into the lumen of the pancreaticobiliary ductal system, like the endoscope used for a lumen having a large diameter, observation and treatment with the treatment instrument of the inside of the lumen can be simultaneously performed, and further, once the insertion part is inserted into the lumen, the treatment instrument can easily be inserted and put in the lumen without using the guide wire or the like by only passing the treatment instrument through the treatment instrument passage channel. Therefore, there is not such a disadvantage as described above.

However, in order to make the insertion part capable of being inserted into the lumen of the pancreaticobiliary ductal system, it is only allowed to increase the diameter of the treatment instrument passage channel to about 2 mm at most. Therefore, there is the problem that the treatment instrument having an outer diameter of about 3 to 4 mm cannot be passed through the treatment instrument passage channel, unlike the stent delivery system.

The present invention has been made in view of these circumstances, and an object of the present invention is to provide an endoscopic device that makes it possible to insert the treatment instrument together with the insertion part into such a narrow lumen as the pancreaticobiliary ductal system easily without inserting the treatment instrument passage channel therein.

In order to achieve the object, an endoscopic device according to the present invention is an endoscopic device provided with an observation window in a distal end face of an insertion part tended to be inserted into a subject, an observational direction from the observation window being substantially identical with a longitudinal axis of the insertion part the endoscope comprising: a concave guide surface disposed in a distal lateral portion of the insertion part so as to extend along the longitudinal direction of the insertion part; and a bending part configured to be bendable with the guide surface inside, wherein a treatment instrument fed from a distal end exit of a treatment instrument passage disposed closer to a proximal end of the insertion part than the guide surface is guided along the guide surface when the bending part is bent.

According to the present invention, when the bending part of the insertion part of the endoscope (endoscopic device) is bent, the treatment instrument fed from the distal end exit of the treatment-instrument passage is guided along the guide surface. For example, when the guide surface is inserted into the lumen of the pancreaticobiliary ductal system and then the treatment instrument is fed from the distal end exit of the treatment-instrument passage with the bending part bent, the treatment instrument is guided by the guide surface, thereby being inserted into the lumen of the pancreaticobiliary ductal system. Therefore, it becomes possible to insert a treatment instrument easily into even a lumen in which the guide surface has been inserted.

In the present invention, it is preferred that the guide surface is provided with a plurality of guide surfaces corresponding to a plurality of treatment instruments different in outer diameter, respectively. According to this aspect, it is possible to guide treatment instruments different in outer diameter more fitly along the guide surfaces corresponding to the outer diameters.

In the present invention, it is preferred that a first groove portion constituting a first guide surface having a first radius of curvature is provided and a second groove portion constituting a second guide surface having a second radius of curvature smaller than the first radius of curvature are provided within the first groove portion, the first guide surface and the second guide surface constituting the guide surface. The present invention shows the aspect where the guide surface is provided with a plurality of guide surfaces corresponding to a plurality of treatment instruments different in outer diameter.

In the present invention, it is preferred that the guide surface is provided within a range from the distal end exit of the treatment instrument passage to the distal end face of the insertion part so as to extend along the longitudinal direction of the insertion part and a covering member for guiding the treatment instrument fed from the distal end exit of the treatment instrument passage to a side on which the guide surface is formed is disposed so as to cover the guide surface. According to this aspect, the treatment instrument fed from an opening portion (the distal end exit) of the treatment-instrument passage securely guided to the guide surface of a treatment-instrument guiding portion insertion part) by the cover member.

In the present invention, it is preferred that a proximal end of the covering member is fixed to the distal end exit of the treatment instrument passage in a cantilever manner.

In the present invention, it is preferred that the guide surface is provided with an engaging member engaged with a distal end of the covering member. According to this aspect, it is possible to hold the covering member on the guide surface with the engaging member when the insertion part of the endoscope (endoscopic device) is inserted into an intended site in a body cavity.

In the present invention, the treatment instrument passage may be disposed inside the insertion part of the endoscope (endoscopic device) or in an insertion aid through which the insertion part is inserted to be guided.

In the present invention, the insertion aid may have a groove portion constituting the treatment instrument passage, the groove portion being formed on an inner wall surface of a passage through which the insertion part is inserted so as to extend along a longitudinal direction of the passage.

In the present invention, the insertion aid may be provided with a first passage through which the insertion part is inserted and a second passage constituted as the treatment instrument passage.

According to the present invention, it becomes possible to insert a treatment instrument together with the insertion part into such a narrow lumen as the pancreaticobiliary ductal system easily without inserting the treatment-instrument passage channel therein.

In the present invention, it is preferred that a cross-section of a portion where the concave guide surface has been formed, which is perpendicular to the longitudinal direction of the insertion part, is a cross-section having the shape of an arc having a central angle of 180 degrees or more with respect to a central axis of the insertion part, and operation wires for bending the bending part are passed through both ends of the cross-section.

According to the present invention, the bending part can be bent with the guide surface inside by simultaneously pulling these two operation wires passed through both the ends, and the bending part can be bent in a direction perpendicular to the guide surface by pulling one of the two operation wires and loosening the other.

### BRIEF DESCRIPTION OF THE DRAWINGS

FIG. 1 is an overall view showing an embodiment of an endoscopic device according to the present invention;

FIG. 2 is an enlarged perspective view showing an insertion part on a distal end side of the endoscopic device;

FIG. 3 is a front view showing a distal end face of the insertion part on the distal end side:

FIG. 4 is a perspective view showing a bending structure (joint ring) of a bending part of the endoscopic device;

FIG. 5 is an illustration used for describing a function of the endoscope (endoscopic device) in FIG. 1 used in performing an operation on the pancreaticobiliary ductal system;

FIG. 6 is a perspective view showing an aspect of the endoscopic device provided with an assistive member to guide a treatment instrument fed from a treatment instrument exit securely into a treatment instrument guide groove of the insertion part on the distal end side;

FIG. 7 is a front view showing the distal end face of the insertion part on the distal end side showing an aspect of the endoscopic device in which, as the treatment instrument guide groove of the insertion part on the distal end side, a plurality of treatment instrument guide grooves corresponding to treatment instruments having different diameters have been formed; and

FIG. 8 is a perspective view of the distal insertion part showing an aspect of the endoscope (endoscopic device) which is not provided with a treatment instrument passage channel.

### DETAILED DESCRIPTION OF THE PREFERRED EMBODIMENTS

Hereinafter, a preferred embodiment of the present invention will be described with reference to the accompanying drawings.

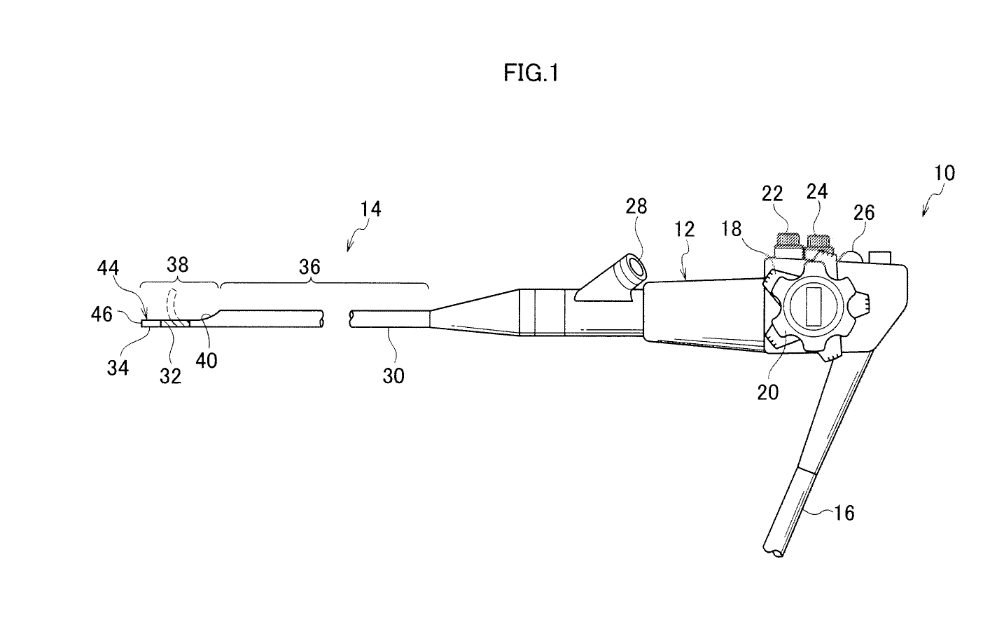

FIG. 1 is an overall view showing an embodiment of an endoscopic device according to the present invention. An endoscope (endoscopic device) 10 in FIG. 1 is an endoscope functioning mainly as a cholangioscope for observing the lumen of the biliary tract or the pancreatic duct (pancreaticobiliary ductal system) and as an insertion aid for inserting various treatment instruments into the lumens of these ducts, and the endoscope 10 is provided with an operation part 12, an insertion part 14 connected with the operation part 12 and intended to be inserted into a body cavity, and a universal cable 16 extended from the operation part 12. At the time of operation, the endoscope 10 is used in connection with an image processor, a light source device, and an air-supply and water-supply device (which are not shown) via the universal cable 16.

The operation part 12 is a unit that grasped by an operator to perform various operations, and the operation part 12 is provided with angle knobs 18, 20 and various buttons intended to be pressed to operate, such as an air-supply and water-supply button 22, a suction button 24, and a shutter button 26. The operation part 12 is also provided with a treatment instrument entrance 28 (opening) for inserting various treatment instruments into a treatment instrument passage channel passing through the insertion part 14.

The insertion part 14 is composed of a flexible part 30 having flexibility, a bending part 32 that bends according to the operation of the angle knobs 18, 20, and a hard distal end portion 34 in this order from the operation part.

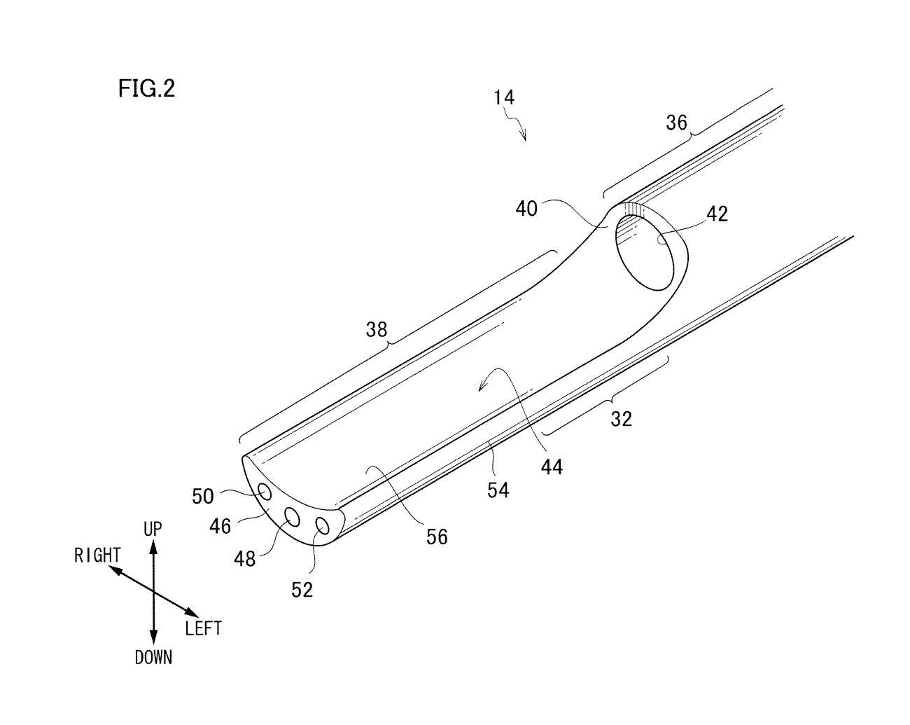

The insertion part 14 is also divided into a proximal insertion part 36 having a substantially-cylindrical-surface-like shape and a distal insertion part 38 having a substantially-crescent shape in cross-section perpendicular to the longitudinal axis of the insertion part 14 (for details, see FIG. 2 described later), and the bending part 32 and the distal end portion 34 are disposed within the range of the distal insertion part 38. The distal insertion part 38 is made thinner than the proximal insertion part 36, and therefore can easily be inserted into the lumen of the pancreaticobiliary ductal system, and, at the time of operation, the proximal insertion part 36 is inserted into the duodenum, and the distal insertion part 38 is inserted into the lumen of the pancreaticobiliary ductal system.

In an interface 40 which is a border area between the proximal insertion part 36 and the distal insertion part 38, a treatment instrument exit 42 (for details, see FIG. 2 described later) which is an opening of the treatment instrument passage channel passing through the insertion part 14 is formed, so that when a treatment instrument is inserted through the treatment instrument entrance 28 of the operation part 12, the treatment instrument is passed through the treatment instrument passage channel and fed from the treatment instrument exit 42, and, as described later, guided by a treatment instrument guide groove 44 of the distal insertion part 38 and placed so as to project forward beyond the distal end portion 34.

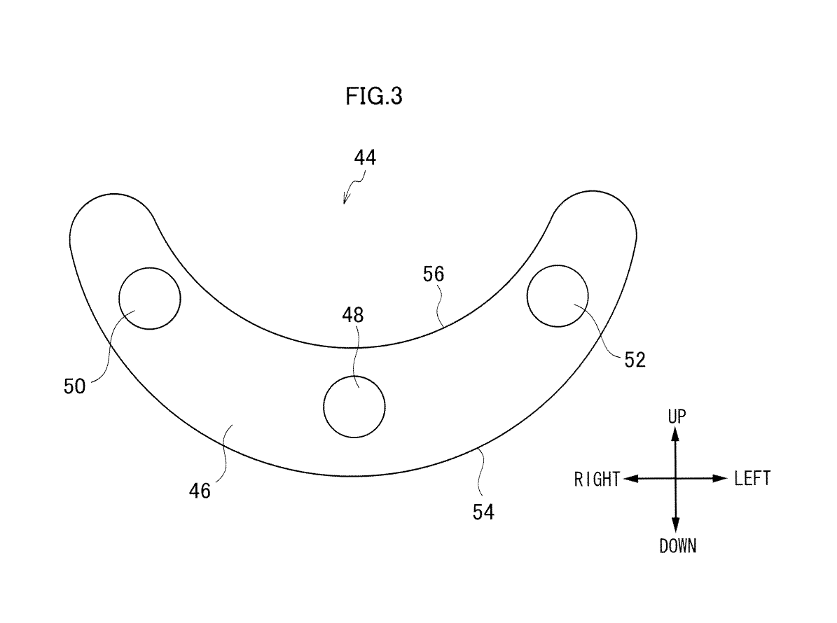

Further, the distal end portion 34 has a built-in imaging device provided with a lens and a solid-state imaging element, and a distal end face 46 of the distal insertion part 38 (a distal end face 46 of the insertion part 14) is provided with an observation window 48 for observation (for details, see FIG. 2, FIG. 3) for capturing subject light into the imaging device. An image captured by the imaging device is inputted as an image signal in the image processor connected with the universal cable 16, subjected to required image processing, and displayed on a monitor. Thus, the operator can observe the inside of a body cavity in which the distal insertion part 38 has been inserted. Further, when the shutter button 26 of the operation part 12 is pressed, an image captured by the imaging device at that time is recorded and stored in the image processor.

As shown in FIGS. 2 and 3, the distal end face 46 of the distal insertion part 38 is provided with an illumination window 50 for emitting illuminating light for illuminating an observation object to be imaged by the imaging device, and an air-supply and water-supply outlet 52 (injection nozzle) for spraying cleaning water or gas to the observation window 48 to clean the observation window. The illumination light emitted from the illumination window 50 is supplied from the light source device connected with the universal cable 16 through a light guide. The cleaning liquid or gas sprayed from the air-supply and water-supply outlet 52 is supplied from the air-supply and water-supply device connected with the universal cable 16 through an air-supply and water-supply channel, and gas is sprayed from the air-supply and water-supply outlet 52 toward the observation window 48 when a hole provided on the air-supply and water-supply button 22 of the operation part 12 is closed, and cleaning liquid is sprayed from the air-supply and water-supply outlet 52 toward the observation window 48 when the air-supply and water-supply button 22 is pressed.

Next, the configuration of the distal insertion part 38 of the endoscope 10 described above will be described in detail. FIG. 2 is an enlarged perspective view showing the distal insertion part 38, and FIG. 3 is a front view showing the distal end face 46 of the distal insertion part 38 (the insertion part 14). As shown in FIGS. 2 and 3, the distal insertion part 38 is extended from the proximal insertion part 36 having a substantially-cylindrical-surface-like shape, and has a substantially-crescent shape in cross-section. At the top of the distal insertion part 38 on the proximal side, the treatment instrument exit 42 formed in the interface 40 between the distal insertion part 38 and the proximal insertion part 36 is located, so that the treatment instrument inserted from the treatment instrument entrance 28 of the operation part 12 and fed from the treatment instrument exit 42 is placed on an upper face side of the distal insertion part 38.

Here, the distal insertion part 38 has a plane-symmetrical shape with respect to a plane of symmetry in a predetermined direction including the central axis of the proximal insertion part 36 (the longitudinal axis of the insertion part 14), and the treatment instrument exit 42 is formed so as to be positioned such that the central axis (the central axis of the treatment instrument passage channel) is included in the plat symmetry. The direction of an axis perpendicular to the longitudinal axis of the insertion part 14 and included in the plane of symmetry is defined as a vertical direction, and the direction perpendicular to the vertical direction is defined as a lateral direction. Further, as shown in FIGS. 2 and 3, one direction, of the vertical direction, of a face of the distal insertion part 38 in which the treatment instrument guide groove 44 is formed is defined as an upward direction, and the other direction is defined as a downward direction, and one direction of the lateral direction to the left of the distal insertion part when viewed from the operation part 12 is defined as a leftward direction, and the other direction is defined as a rightward direction.

The distal insertion part 38 has a whole external surface formed of a lower external surface 54, the distal end face 46, and an upper external surface 56.

The lower external surface 54 forms a lower surface of the distal insertion part 38, is connected with a cylindrical-surface-like external surface of the proximal insertion part 36, and has a shape following a cylindrical surface coaxial with and the same diameter as an external surface of the proximal insertion part 36. It should be noted that the lower external surface 54 may have a shape following a cylindrical surface having a smaller diameter than the external surface of the proximal insertion part 36.

The distal end face 46 is also a face serving as the most distal end of the whole insertion part 14, and on the distal end face 46, as described above, the observation window 48, the illumination window 50, and the air-supply and water-supply outlet 52 are located. It should be noted that a spray nozzle for spraying cleaning liquid or gas to the observation window 48 is attached to the air-supply and water-supply outlet 52.

The upper external surface 56 forms an upper surface of the distal insertion part 38, and connected with the lower external surface 54. This upper external surface 56 is formed into a concave shape that is curved like an arc along a substantially-cylindrical surface, that is, a shape following a cylindrical surface having its axis at a different position from the cylindrical surface followed by the lower external surface 54, and the treatment instrument guide groove 44 extending along a long axis of the insertion part 14 is formed of this concave upper external surface 56. A treatment instrument fed from the treatment instrument exit 42 is guided (led) along the treatment instrument guide groove 44 by the treatment instrument guide groove 44 formed of the upper external surface 56. It should be noted that the shape of an inner wall surface of the treatment instrument guide groove 44 is not limited to the arc shape, and may be an angular C or U shape in cross-section, or may be another shape.

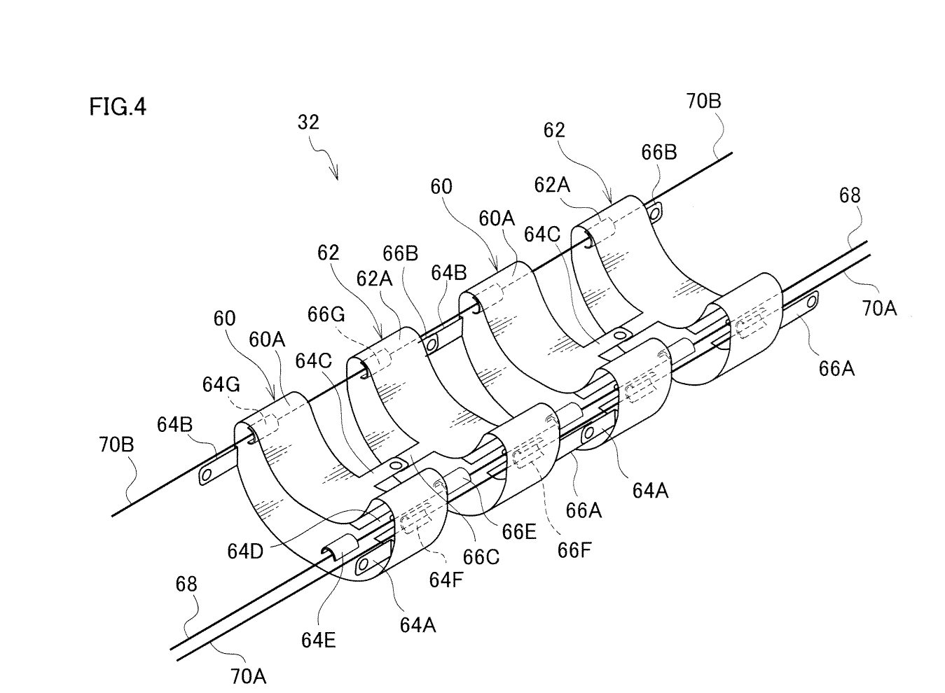

The distal insertion part 38 also has the bending part 32 that is operated to bend according to the angle knobs 18, 20 of the operation part 12, as described above. A bending structure of the bending part 32 will be illustrated in FIG. 4.

As shown in FIG. 4, the bending part 32 has a plurality of first joint rings 60 and second joint rings 62 joined alternately along the long axis (in a longitudinal direction) of the insertion part 14. Though FIG. 4 shows only two first joint rings 60 and two second joint rings 62, a desired number of first joint rings 60 and second joint rings 62 can be joined. The first joint rings 60 and the second joint rings 62 are entirely covered with a casing, and the casing forms the lower external surface 54 and the upper external surface 56 of the distal insertion part 38, and each of the first joint rings 60 and each of the second joint rings 62 have annular members 60A and 62A, each having substantially the same cross-sectional shapes as the external surface formed as the lower external surface 54 and the upper external surface 56.

The first joint ring 60 has a left hinge part 64A and a right hinge part 64B projecting forward on both left and right sides of the annular member 60A, and has an upper hinge part 64C and a lower hinge part 64D projecting backward on both upper and lower sides of the annular member 60A.

On the other hand, the second joint ring 62 has a left hinge part 66A and a right hinge part 66B projecting backward on both left and right sides of the annular member 62A, and has an upper hinge part 66C and a lower hinge part 66D (not shown) projecting forward on both upper and lower sides of the annular member 62A.

The left hinge part 64A and the right hinge part 64B of the first joint ring 60 are pivotally coupled by a coupler, such as a rivet, with the left hinge part 66A and the right hinge part 66B of the second joint ring 62 located just in distal end direction of this first joint ring 60. On the other hand, the upper hinge part 64C and the lower hinge part 64D of the first joint ring 60 is pivotally coupled by a coupler, such as a rivet, with the upper hinge part 66C and the lower hinge part 66D of the second joint ring 62 located just in operation part direction of this first joint ring 60. Thereby, the second joint ring 62 located just in distal end direction of the first joint ring 60 is supported so as to be vertically swingable, and the second joint ring 62 just in operation part direction the first joint ring 60 is supported so as to be laterally swingable. Therefore, the bending part 32 has a four-directionally, vertically and laterally, bendable structure.

Further, lower wire receiving parts 64E and 66E, left wire receiving parts 64F and 66F, and right wire receiving parts 64G and 66G, each of which has a passage hole that an operation wire passes through, are formed so as to project from inner surfaces of each first joint ring 60 and each second joint ring 62, respectively.

The lower wire receiving parts 64E and 66E of each first joint ring 60 and each second joint ring 62 are aligned in the longitudinal direction of the insertion part 14, a lower operation wire 68 is passed through these lower wire receiving parts 64E and 66E, and a distal end of the lower operation wire 68 is fixed to the first joint ring 60 or the second joint ring 62, whichever is located at the most distal end.

The left wire receiving parts 64F and 66F and the right wire receiving parts 64G and 66G of each first joint ring 60 and each second joint ring 62 are aligned in the longitudinal direction of the insertion part 14 like the lower wire receiving parts 64E and 66E, and are provided above pivoting points of the left hinge parts 64A and 66A and the right hinge parts 64B and 66B. A left operation wire 70A and a right operation wire 70B are passed through the left wire receiving parts 64A and 66F, and the right wire receiving parts 64G and 66G and distal ends of the operation wires 70A and 70B are fixed to the first joint ring 60 or the second joint ring 62, whichever is located at the most distal end.

It should be noted that a cross-section perpendicular to the long axis of the distal insertion part 38 is a cross-section having the shape of an arc having a predetermined central angle (for example, 180° or more) with respect to a central axis (the long axis of the insertion part 14) of the proximal insertion part 36, and the left operation wire 70A and the right operation wire 70B are passed through both end portions of the arc-shaped cross-section.

On the other hand, each of the operation wires 68, 70A, and 70B is passed through the insertion part 14 and connected with a wire pulling mechanism within the operation part 12. The wire pulling mechanism has a mechanism to pull or loosen each of the operation wires 68, 70A, and 70B in conjunction with a rotating operation of each of the angle knobs 18 and 20 of the operation part 12, and the wire pulling mechanism pulls or loosens each of the operation wires 68, 70A, and 70B so that the bending part 32 bends in accordance with an operation of each of the angle knobs 18 and 20.

For example, when the angle knob 18 is rotated in a predetermined direction, the wire pulling mechanism pulls both the left operation wire 70A and the right operation wire 70B and loosens the lower operation wire 68. This causes the bending part 32 to bend upward, that is, to the side of the treatment instrument guide groove 11 (the upper external surface 56). When the angle knob 18 is turned in the opposite direction, the wire pulling mechanism pulls the lower operation wire 68 and loosens both the left operation wire 70A and the right operation wire 70B. This causes the bending part 32 to bend downward, that is, to the side of the lower external surface 54.

On the other hand, when the angle knob 20 is rotated in a predetermined direction, the wire pulling mechanism pulls the left operation wire 70A and loosens the right operation wire 70B. This causes the bending part 32 to bend leftward. When the angle knob 20 is rotated in the opposite direction, the wire pulling mechanism pulls the right operation wire 70B and loosens the left operation wire 70A. This causes the bending part 32 to bend rightward.

It should be noted that the lower operation wire 68 is not essential, and it is possible to bend the bending part 32 vertically by pushing and pulling operation of both the left operation wire 70A and the right operation wire 70B. Further, in a usage aspect of the endoscope 10 described later, it is not essential for the bending part 32 to bend laterally, and the wire pulling mechanism does not have to be provided with a mechanism to pull one of the left operation wire 70A and the right operation wire 70B and loosen the other. In this case, it is also possible to eliminate the left operation wire 70A and the right operation wire 70B and provide only the lower operation wire 68 so that only the lower operation wire 68 is used to bend the bending part 32 vertically. Furthermore, it is only necessary for the bending part 32 to be bendable at least upward (to the side of the guide groove) in relation to a straight state thereof.

Further, it is not necessary for the bending part 32 to be completely included in the distal insertion part 38, and the bending part 32 may be partially included in the proximal insertion part 36.

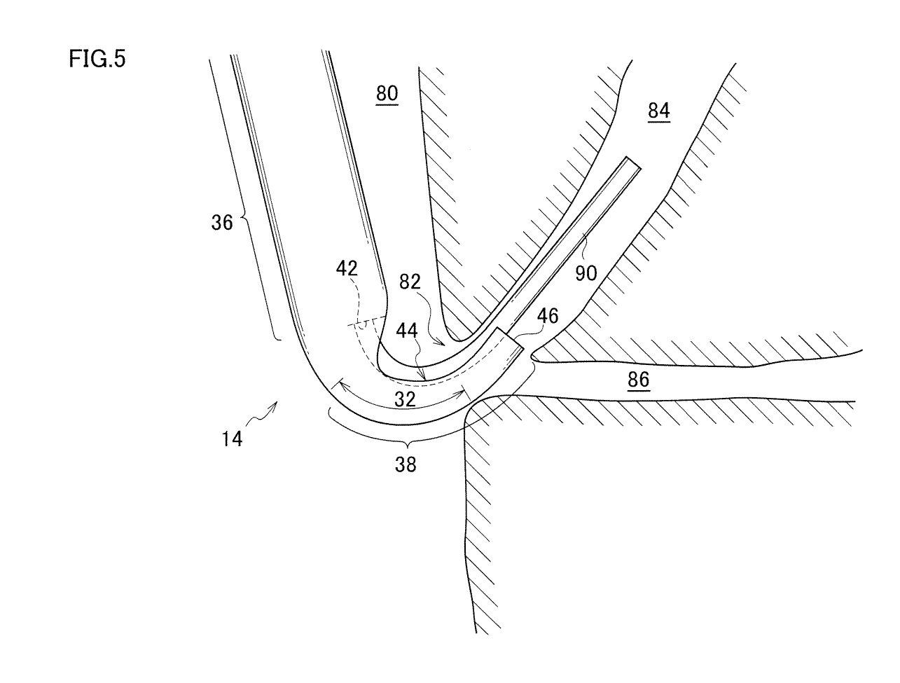

The function of the endoscope 10 thus configured when used in an operation on the pancreaticobiliary ductal system will be described with reference to FIG. 5. During the operation, the insertion part 14 of the endoscope 10 is inserted through the patient's mouth, and the distal end of the insertion part is passed through the esophagus and the stomach into the vicinity of a papilla of Vater (major duodenal papilla) 82 located in a duodenum 80.

Next, the distal end of the distal insertion part 38 is inserted through an opening of the papilla of Vater 82 into the lumen of a bile duct (common bile duct) 84 or a pancreatic duct 86. Here, it is assumed that the distal end of the distal insertion part 38 is inserted into the bile duct 84. Thus, an image of the inside of the bile duct 84 observed through the observation window 48 located in the distal end face 46 of the distal insertion part 38 is captured, and observed on a monitor or the like.

Then, at least when a predetermined treatment instrument 90 is inserted into the bile duct 84, the operator holds the endoscope 10 with a part of the distal end of the distal insertion part 38 interposed in the bile duct 84, as shown in FIG. 5. Further, at this time, the angle knob 18, 20 or the like of the operation part 12 is operated so as to hold the endoscope 10 with the bending part 32 bent to the side of the treatment instrument guide groove 44 (upward).

Next, the operator inserts the treatment instrument 90 through the treatment instrument entrance 28 of the operation part 12. After the distal end of the treatment instrument 90 is fed from the treatment instrument exit 42 accordingly, the treatment instrument 90 abuts on the treatment instrument guide groove 44 of the distal insertion part 38 at least at the bending part 32. Then, the treatment instrument 90 slides forward on the treatment instrument guide groove 44, turns toward the papilla of Vater 82 at the bending part 32, and slides continuously along the treatment instrument guide groove 44 to enter the bile duct 84 through the opening of the papilla of Vater 82. In this manner, the distal end of the treatment instrument 90 is guided to the distal end of the treatment instrument guide groove 44, and thereafter, with transition of part of the treatment instrument 90 guided by the treatment instrument guide groove 44 to the proximal end side thereof, the distal end of the treatment instrument 90 is delivered forward beyond the distal end (the distal end face 46) of the distal insertion part 38 so as to move further forward in the bile duct 84.

Thus, once the operator inserts the distal insertion part 38 of the endoscope 10 through the opening of the papilla of Vater 82, as shown in FIG. 5, and interposes the distal insertion part 38 in the lumen of the pancreaticobiliary ductal system, the operator can easily interpose the treatment instrument 90 in the lumen of the pancreaticobiliary ductal system only by inserting the treatment instrument 90 through the treatment instrument entrance 28 and delivering the same. Further, within the observational field of view in which observation is performed through the observation window 48 of the distal end face 46 of the distal insertion part 38, it is also possible to interpose the treatment instrument 90 at a desired position while watching the observed image on the monitor without performing fluoroscopy or the like.

Further, since the treatment instrument 90 used in the pancreaticobiliary ductal system may be such a large treatment instrument as a stent delivery system having an outer diameter of about 3 to 4 mm, the treatment instrument passage channel of the endoscope 10 has at least a larger diameter than the treatment instrument 90. At this time, if the treatment instrument passage channel is extended to the distal end of the insertion part 14, the outer diameter of the distal end portion of the insertion part 14 becomes too large to be inserted easily into the lumen of the pancreaticobiliary ductal system. On the other hand, in the endoscope 10 according to the embodiment described above, since the proximal insertion part 36 is provided with the treatment instrument passage channel terminating, and the distal insertion part 38 located in front of the proximal insertion part 36 is not provided with the treatment instrument passage channel so as to be narrowed (for example, a maximum width (lateral width) of about 3 mm), it is possible to easily insert the distal end portion of the insertion part 14 into the lumen of the pancreaticobiliary ductal system. In addition, since the distal insertion part 38 is provided with the treatment instrument guide groove 44, it is possible to guide the treatment instrument 90 to the distal end of the insertion part 14 without the treatment instrument passage channel. Further, even if the treatment instrument 90 having a large outer diameter, such as a stent delivery system, is placed along the treatment instrument guide groove 44 of the distal insertion part 38, the distal insertion part 14 is sufficiently thinner than an insertion part provided with the treatment instrument passage channel through which the treatment instrument 90 can be passed so that the distal insertion part 14 can be inserted into the lumen of the pancreaticobiliary ductal system. It should be note that if the distal insertion part 38 is made long (for example, about 10 cm), it also becomes possible to insert the distal insertion part 38 into deep parts of the pancreaticobiliary ductal system to observe the deep part.

Next, a modification of the endoscope 10 described above will be described. It should be noted that in the figures used below, components identical with or similar to those in the endoscope 10 will be marked with the same reference numerals so as not to be described repeatedly.

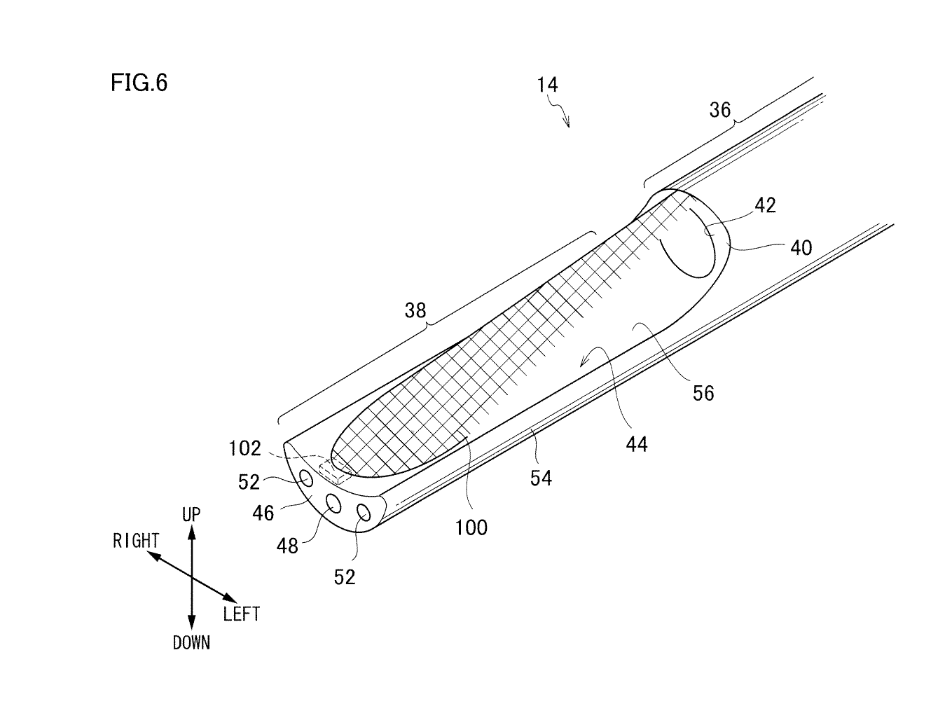

FIG. 6 is a perspective view showing an aspect of the endoscope 10 provided with an assistive member (covering member) 100 to lead a treatment instrument fed from the treatment instrument exit 42 securely into the treatment instrument guide groove 44 of the distal insertion part 38. The distal insertion part 38 in FIG. 6 is provided with, for example, an elastically-deformable thin-plate-like assistive member 100 above the treatment instrument guide groove 44.

The assistive member 100 has a proximal end fixed to the interface 40 above the treatment instrument exit 42 so as to be supported in a cantilever manner such that a distal end of the assistive member 100 is in substantial contact with the vicinity of the distal end of the distal insertion part 38 when a treatment instrument is not fed from the treatment instrument exit 42.

On the other hand, when a treatment instrument is fed from the treatment instrument exit 42, the treatment instrument abuts on the assistive member 100, and is led to the inside of the treatment instrument guide groove 44 so as not to derail from the treatment instrument guide groove 44. The assistive member 100 is swung about a proximal end thereof and deformed so as to pass the treatment instrument between the treatment instrument guide groove 44 and the assistive member 100. Thus, providing the endoscope 10 with the assistive member 100 allows the treatment instrument fed from the treatment instrument exit 42 to be placed securely in the treatment instrument guide groove 44.

It should be noted that when the distal insertion part 38 is inserted from the papilla of Vater 82 into the lumen of the pancreaticobiliary ductal system (for example, into the bile duct 84), as shown in FIG. 5, the assistive member 100 is also inserted into the bile duct 84, and held in substantial contact with the treatment instrument guide groove 44. Therefore, even if the assistive member 100 is not elastic, the assistive member 100 has the function of leading the treatment instrument into the treatment instrument guide groove 44. Therefore, it is only necessary for the assistive member 100 to be a deformable member. Further, a projection 102 that engages with the distal end of the assistive member 100 may be formed, as shown in FIG. 6, on the distal end of the treatment instrument guide groove 44 so as to prevent the distal end of the assistive member 100 from moving away from the treatment instrument guide groove 44 until the distal insertion part 38 is inserted into the bile duct 84.

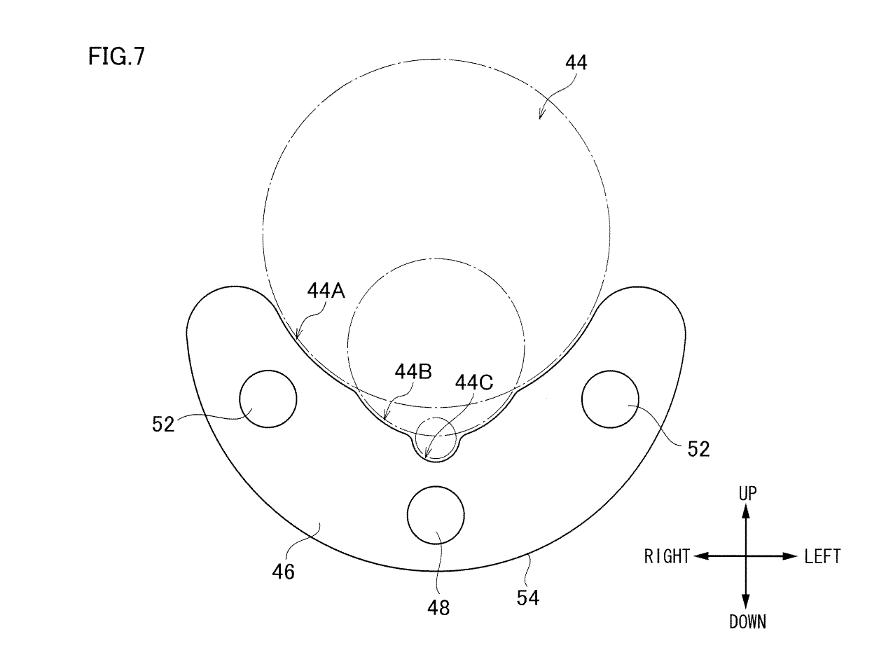

FIG. 7 is a front view of the distal end face 46 of the distal insertion part 38, showing an aspect of the endoscope 10 in which the treatment instrument guide groove 44 of the distal insertion part 38 is formed of a plurality of treatment instrument guide grooves corresponding to treatment instruments having different outer diameters.

The treatment instrument guide groove 44 of the distal insertion part 38 in FIG. 7 is formed of a first treatment instrument guide groove 44A, a second treatment instrument guide groove 44B, and a third treatment instrument guide groove 44C in such a manner that grooves smaller in width (smaller in radius of curvature) than the treatment instrument guide groove 44 is formed within (in the bottom of) the treatment instrument guide groove. The first treatment instrument guide groove 44A is a groove having the largest width (radius of curvature) so as to be engaged with a treatment instrument having the largest outer diameter, and the third treatment instrument guide groove 44C is a groove having the smallest width (radius of curvature) so as to be engaged with a treatment instrument having the smallest outer diameter.

On the other hand, treatment instruments used in operations on the pancreaticobiliary ductal system includes, for example, a stent delivery system for placing a stent at a site of stenosis, a basket for crushing a gallstone, a balloon catheter for bringing out a gallstone or the like, a biopsy instrument for sampling body tissue, and the like. A guide wire for leading the treatment instruments to their destinations is also used in operations on the pancreaticobiliary ductal system. It should be noted that the guide wire is a kind of treatment instrument.

A relatively large treatment instrument, such as a stent delivery system having an outer diameter of about 4 mm, is engaged with and guided by the first treatment guide groove 44A. A treatment instrument having a smaller outer diameter than the stent deliver system, such as a basket, a balloon catheter, or a biopsy instrument having a diameter of about 3 mm, is engaged with and guided by the second treatment instrument guide groove 44B. An extremely narrow treatment instrument, such as a guide wire having a diameter of about 1 mm, is engaged with and guided by the third treatment instrument guide groove 44C.

Thus, when treatment instruments different in outer diameter are used, the treatment instruments are engaged with engageable smallest-radius-of-curvature one of the first to third treatment instrument guide grooves 44A, 44B, and 44C, so that lateral deviation of the treatment instruments is reduced.

It should be noted that though FIG. 7 shows the aspect where the first to third treatment instrument guide grooves 44A, 44B, and 44C are formed, not only two but also three or four grooves may be formed.

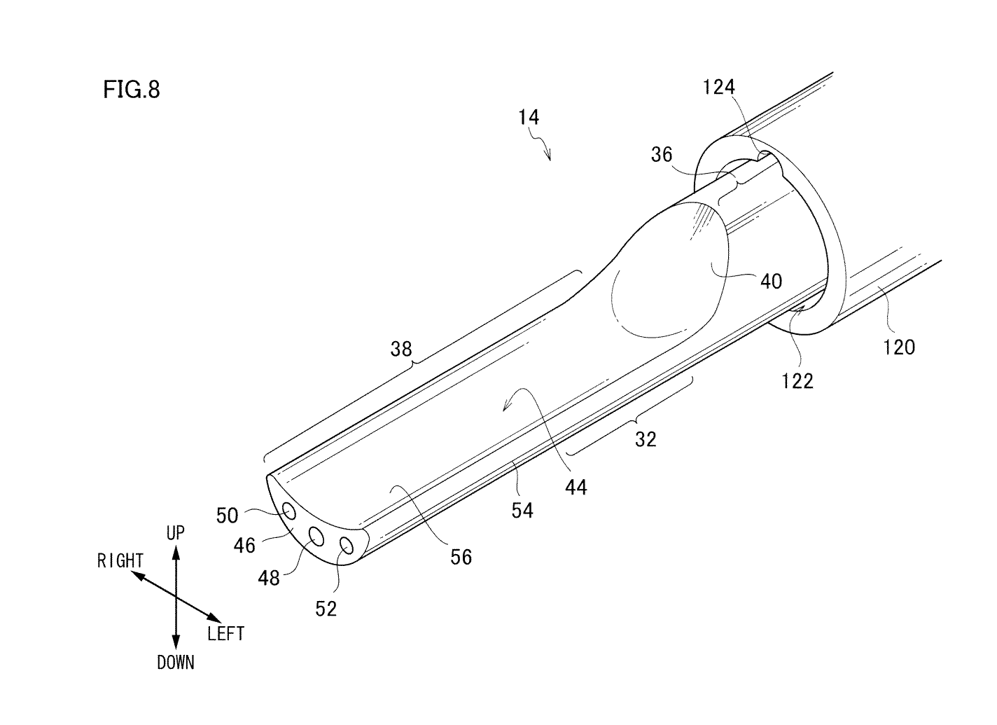

FIG. 8 is a perspective view of the distal insertion part 38, showing an aspect of the endoscope 10 not provided with a treatment instrument passage channel.

The endoscope 10 shown in FIG. 8 is not provided with a treatment instrument passage channel. Therefore, a treatment instrument exit in such an aspect as shown in FIG. 2 is not formed either in the interface 40 between the proximal insertion part 36 and the distal insertion part 38, and the proximal insertion part 36 in FIG. 8 is narrower in diameter than the proximal insertion part 36 in FIG. 2. On the other hand, the distal insertion part 38 in FIG. 8 is configured in the same manner as the distal insertion part 38 in FIG. 2.

When a treatment instrument is used in the endoscope 10 thus configured, an overtube 120 having a treatment instrument passage groove 124 through which the treatment instrument is passed is attached to the insertion part 14 in such a manner that the insertion part 14 is inserted into a lumen 122 of the overtube 120 so as to project the distal end of which the distal insertion part 38 from a distal end of the overtube 120, as shown in FIG. 5.

The treatment instrument passage groove 124 of the overtube 120 is formed in an inner wall of the lumen 122 in a longitudinal direction thereof, so that the treatment instrument inserted through a proximal end of the overtube 120 is fed from the distal end of the overtube 120 along the treatment instrument passage groove 124.

Further, when the overtube 120 is attached to the insertion part, the treatment instrument passage groove 124 is positioned above the proximal insertion part 36 so that the treatment instrument passage groove 124 is aligned with the treatment instrument guide groove 44 of the distal insertion part 38.

According to the endoscope 10 thus configured, with the bending part 32 bent upward (to the side of the treatment instrument guide groove 44) so as to insert the distal insertion part 38 into the bile duct or the like through the papilla of Vater in the same manner as shown in FIG. 5, a treatment instrument is fed from the distal end of the overtube 120 through the treatment instrument passage groove 124 of the overtube 120, and then a distal end of the treatment instrument abuts on the treatment instrument guide groove 44 bent at the bending part 32. The treatment instrument is moved further forward, then turned toward the papilla of Vater at the bending part 32, and continuously moved along the treatment instrument guide groove 44 so as to enter the bile duct 84 or the like through the papilla of Vater 82. Therefore, the treatment instrument can be interposed easily in the lumen of the pancreaticobiliary ductal system in the same manner as the distal insertion part 38 of the insertion part 14 shown in FIG. 2.

It should be noted that instead of providing the overtube 120 with the treatment instrument passage groove 124, a lumen for a treatment instrument to pass through may be formed. Also in the aspect shown in FIG. 8 it is possible to dispose an assistive member similar to the assistive member 100 in FIG. 6 above the treatment instrument guide groove 44. In this case, a proximal end of the assistive member is fixed to the overtube 120 above the treatment instrument passage groove 124. Also, as shown in FIG. 7, the treatment instrument guide groove 44 may be provided with a plurality of treatment instrument guide grooves.

In the embodiments described above, the treatment instrument guide groove 44 is formed over the entire length of the distal insertion part 38, but not necessarily formed over the entire length thereof.

What is claimed is:

1. An endoscopic device provided with an observation window in a distal end face of an insertion part to be inserted into a subject, an observational direction from the observation window being substantially identical with a longitudinal axis of the insertion part, the endoscope comprising:
a concave guide surface disposed in a distal lateral portion of the insertion part so as to extend along a longitudinal direction of the insertion part; and
a bending part configured to be bendable with the guide surface inside, wherein
a treatment instrument fed from a distal end exit of a treatment instrument passage disposed closer to a proximal end of the insertion part than the guide surface is guided along the guide surface when the bending part is bent.

2. The endoscopic device according to claim 1, wherein the guide surface is provided with a plurality of guide surfaces corresponding to a plurality of treatment instruments different in outer diameter.

3. The endoscopic device according to claim 2, wherein the guide surface is provided with a first groove portion constituting a first guide surface having a first radius of curvature and is provided with a second groove portion constituting a second guide surface having a second radius of curvature smaller than the first radius of curvature, the second groove portion being within the first groove portion.

4. The endoscopic device according to claim 1, wherein
the guide surface is provided within a range from the distal end exit of the treatment instrument passage to the distal end face of the insertion part so as to extend along a longitudinal direction of the insertion part; and
a covering member for guiding the treatment instrument fed from the distal end exit of the treatment instrument passage to a side on which the guide surface is formed is disposed so as to cover the guide surface.

5. The endoscopic device according to claim 4, wherein a proximal end of the covering member is fixed to the distal end exit of the treatment instrument passage in a cantilever manner.

6. The endoscopic device according to claim 5, wherein the guide surface is provided with an engaging member engaged with a distal end of the covering member.

7. The endoscopic device according to claim 1, wherein the treatment instrument passage is disposed inside of the insertion part of the endoscope or in an insertion aid through which the insertion part is passed to be guided.

8. The endoscopic device according to claim 7, wherein the insertion aid have a groove portion constituting the treatment instrument passage, the groove portion being formed on an inner wall surface of a passage through which the insertion part is inserted so as to extend along a longitudinal direction of the passage.

9. The endoscopic device according to claim 7, wherein the insertion aid is provided with a first passage through which the insertion part is passed, and a second passage constituting the treatment instrument passage.

10. The endoscopic device according to claim 1, wherein a cross-section of a portion where the concave guide surface has been formed, which is perpendicular to the longitudinal direction of the insertion part, is a cross-section having the shape of an arc having a central angle of 180 degrees or more with respect to a central axis of the insertion part, and operation wires for bending the bending part are passed through both ends of the cross-section.
